(19) United States
(12) Patent Application Publication
(10) Pub. No.: US 2008/0306344 A1
(43) Pub. Date: Dec. 11, 2008

(54) Surgical retractor securing device with capture assembly

(76) Inventors: Anthony Looper, Lake Zurich, IL (US); Stacey Hoebel, Palatine, IL (US); Matthew Harris, Twin Lakes, WI (US); Chris Hernandez, Gilroy, CA (US); Udo Wiedenmaier, Kirkwood, MO (US)

Correspondence Address: Kimberly Luna;Allegiance Corporation, 1430 Waukegan Road, McGaw Park, IL, 60085, US

(21) Appl. No.: 11/810,500

(22) Filed: Jun. 6, 2007

(51) Int. Cl. A61B 1/32

(52) U.S. Cl. 600/206

(57) ABSTRACT

A resilient compressive surgical retractor securing device with capture assembly includes a compressive securing device that compressively joins the resilient compressive surgical retractor securing device with capture assembly to a surgical accessory bar of a halo style head fixation device and a capture assembly that releasably retains the elasticized band of a scalp hook retractor. The compressive securing device is formed with an arm that is configured to form a neck for receiving the surgical accessory bar or other external support between the arm and the compressive securing device. The arm may be resilient or non-resilient, and may employ releasable closures to close the neck. The capture assembly may include a pivotable cam that releasably retains the elasticized band of a scalp hook retractor by compressing the band between the cam and the surgical accessory bar or other external support.

### TECHNICAL FIELD

The invention relates to the field of surgical instruments, and more particularly, to a surgical retractor securing device with capture assembly that is adapted to be releasably secured to a surgical accessory bar and is effective for releasably retaining surgical retractors during operative procedures.

### BACKGROUND OF THE INVENTION

Surgical retractors designed to retract tissues during surgery have been in use for many years. These retractors may be generally classified into two types; those which are designed to be held free and manipulated by an assistant during surgery, and those that are attached to the patient, the operating table or other external support. The latter class is styled in the art as self-retaining retractors. A common style of self-retaining retractor has an annular ring that supports substantially opposing retractors around an incision. The substantially opposing retractors are locked into place on the annular ring, commonly by ratchet devices, or with a slide and thumbscrew assembly. These retractors are commonly designed to produce considerable and continuous pressure to retract strong tissues, such as abdominal tissues. Alternatively, a retractor may be fixed to the skin of the patient. Also, a retractor may be attached to the surgical drapes which cover the patient and operative field during surgery.

Intracranial neurosurgery makes particular demands for the retraction of human tissue to allow the surgeon maximum access and stability of the surgical field. Due to the extremely delicate nature of the surgery, the surgical field, that is, the patient's head, must be held entirely immobile throughout the often lengthy procedure. Secondly, various tissues of differing tensile strengths, ranging from relatively strong scalp tissue to exceedingly delicate brain structures, must be retracted to allow surgical access during the procedure. The immobilization of the patient's head is commonly achieved by a halo style fixation device, such as that described by Dinkler et al., U.S. Pat. No. 5,529,358. Fixation devices, such as the screws disclosed in Dinkler et al., '358, attach the halo device to the bone of the patient's cranium, achieving positive fixation. The halo portion of the device surrounds the cranium, and may be adapted to hold various appliances during surgery, such as retractors, as further disclosed by Dinkler '358.

A particular demand of intracranial neurosurgery is the need to retract a flap of the patient's scalp away from the opening to be made through the bone of the patient's cranium. An incision is made about the sides and posterior border through the scalp, comprising the planned flap, and the flap is reflected away from the underlying bone, maintaining a hinge-like attachment to the scalp, such that blood supply is preserved to the flap. The scalp flap, remaining attached at its border, therefore has the tendency to fall back across the planned bone incision site, unless it is dependably retracted. The traditional means for accomplishing this intraoperative retraction is through the use of scalp hook retractors, which are devices well known in the art. These scalp hook retractors comprise a proximal sharp hooked portion made of a rigid material, such as surgical steel, attached to a distal elasticized band. The traditional application of these scalp hook retractors in the art has been to evert the scalp flap away from the planned bone incision site, placing the proximal sharp hooked portion of the scalp hook into the underside edge of the scalp flap, and stretching the elasticized band of the scalp hook retractor away from the planned bone incision site. This action imparts a tensile stretch and pulls the edge of the scalp flap away from the planned bone incision site, and the stretch is maintained by looping or tying the elasticized band of the scalp hook retractor to the halo portion of the cranial fixation device. This allows the elasticized band of the scalp hook retractor to exert a continuous, but gentle, force on the edge of the scalp flap, retaining it away from the surgical site.

While effectively maintaining the scalp flap away from the surgical site, these means of attaching the elasticized band of the scalp hook retractor to the halo device pose several recurring problems. First, the attachment of the elasticized band of the scalp hook retractor to the halo device by tying or looping creates a potentially insecure attachment to the halo device, such that the elasticized portion of the scalp hook retractor may slip during surgery. Owing to the extremely delicate nature of the surgery, such sudden motion in the surgical field can have disastrous consequences for the patient. Second, the nature of a halo style fixation device is that it provides a plurality of potential positions about the patient's skull such that the point of fixation of the scalp hook retractor, and thereby its vector of force, may be radially varied. However, to achieve such radial adjustment, repositioning the scalp hook retractor requires untying or unlooping the elasticized band of the scalp hook and then re-tying or re-looping the elasticized band, a cumbersome and time consuming procedure, particularly if a plurality of scalp hooks need to be readjusted. Third, fixation by tying or looping makes it difficult for the surgeon to effectively select the amount of tension desired, as the length, and therefore tension, of the elasticized material will often change while manually tying or looping the elasticized material.

Many of the problems associated with the means of attaching the elasticized band of the scalp hook retractor to the halo device described above have been addressed by Bass et al., U.S. Pat. No. 6,616,604. However, Bass et al., '604, employs an adjustable securing apparatus whereby a manual compression adjuster is utilized to tighten and loosen the compression exerted by the adjustable securing apparatus upon a surgical accessory bar. Thus, if a surgeon needs to reposition the retractor and its associated adjustable securing apparatus to a different point on the surgical accessory bar he or she will have to manually loosen the compression adjuster, reposition the adjustable securing apparatus, and retighten the compression adjuster. This can be a burdensome, time consuming, and potentially life threatening task, especially if time is a critical factor.

Accordingly, there remains an unfulfilled need to provide a device for anchoring surgical retractors in position, satisfying the need for a secure attachment, yet one that is capable of being released and repositioned quickly and easily by the surgeon.

### SUMMARY OF INVENTION

In its most general configuration, the present invention advances the state of the art with a variety of new capabilities and overcomes many of the shortcomings of prior devices in new and novel ways. In its most general sense, the present invention overcomes the shortcomings and limitations of the prior art in any of a number of generally effective configurations. The instant invention demonstrates such capabilities and overcomes many of the shortcomings of prior methods in new and novel ways.

The present invention is a surgical retractor securing device with capture assembly. The surgical retractor securing device with capture assembly is adapted to be releasably secured to a surgical accessory bar having a surgical accessory bar width, or other external support, and operative to retain an elasticized band of a scalp hook retractor while maintaining a continuous but adjustable tension on the scalp or other tissue being retracted.

The surgical retractor securing device with capture assembly includes a securing device for releasably joining the surgical retractor securing device with capture assembly to the surgical accessory bar. The securing device has a proximal end, a distal end, and an arm adjacent to the proximal end. The arm may be configured to form a neck between the arm and the distal end. The width of the neck is smaller in at least one dimension than the surgical accessory bar width and the neck has a neck opening less than the surgical accessory bar width.

Accordingly, the arm must be displaced from a rest position to allow the surgical accessory bar to pass through the neck opening into the neck. The arm therefore applies compressive force to hold the surgical accessory bar in the neck when the surgical accessory bar is in a securing position.

The surgical retractor securing device with capture assembly also includes a capture assembly for releasably retaining the elasticized band and is rotatably attached to the securing device.

The arm may be formed integrally to the securing device and compressive force is therefore created by the resilience of the arm, or the arm may be releasably attached to the securing device, and compressive force may therefore be created by a force producing element applying force to the arm.

In addition to force applied by the arm, additional means may be employed to retain the apparatus on the surgical accessory bar, such as a releasable neck closure attachable to the arm. Such a releasable neck closure may be a releasable gate or a releasable shackle, and would have an open position and a closed position to close the neck opening and secure the surgical accessory bar in the neck. The releasable neck closure is opened to mount or dismount the apparatus on the surgical accessory bar, and when closed, the releasable neck closure helps retain the apparatus in operative position.

In a preferred embodiment, the capture assembly is formed with a capture pivot assembly receiver and the securing device is formed with a device pivot assembly receiver. The capture assembly is rotatably attached to the securing device by a pivot assembly, and the capture assembly may be formed with at least one gripping cam.

It may be desirable to limit the degree of rotation of the capture assembly around the securing device, and therefore in one embodiment, the securing device has a stop pin and the capture assembly has a stop pin limiter. Cooperation between the stop pin and the stop pin limiter limits angular rotation of the capture assembly about the securing device to less than a predetermined degree of motion, which in various preferred embodiments may be less than 45 degrees of rotational motion. In one particular embodiment, the stop pin limiter is a female receiver sized slightly somewhat larger than a male stop pin. Thus, varying the degree of oversize between these two structures allows the fabricator to vary the degree of angular rotation allowed. In some embodiments, the stop pin may be attached to the securing device by means of the stop pin being received into a stop pin receiver, while in other embodiments, the stop pin may be integrally formed with the securing device.

The capture assembly may be a gripping cam that releasably retains the elasticized band between the surgical accessory bar and the gripping cam. The gripping cam may be rotatably attached to the securing device by the pivot assembly passing through a portion of the capture pivot assembly receiver and the device pivot assembly receiver. In some embodiments, the gripping cam may include a high friction gripping surface releasably retaining the elasticized band.

As would also be well known to one skilled in the art, many different functional methods may be employed in different embodiments to secure the capture assembly to the securing device in a rotable manner. In one embodiment, the pivot assembly may be formed of a fastener and stud type arrangement, in which a cam pivot pin is formed with an enlarged head, a tool engager, and a threaded stud engager, and where the cam pivot pin passes through a portion of the capture pivot assembly receiver. To secure the cam pivot pin, a stud with a threaded receiver may pass through a portion of the device pivot assembly receiver in a direction retrograde to the cam pivot pin and the threaded stud engager releasably mates to the threaded receiver.

In another preferred embodiment, the cam pivot pin is a shoulder bolt releasably attachable to the device pivot assembly receiver. The shoulder bolt would ensure that cam pivot pin is tightened only enough to lightly hold the gripping cam against the compressive securing device, and thus would prevent over tightening.

The system of the instant invention enables a significant advance in the state of the art. The instant invention is, in addition, widely applicable to a large number of applications. Variations, modifications, alternatives, and alterations of the various preferred embodiments may be used alone or in combination with one another, as will become more readily apparent to those with skill in the art with reference to the following detailed description of the preferred embodiments and the accompanying figures and drawings.

### BRIEF DESCRIPTION OF THE DRAWINGS

Without limiting the scope of the present invention as claimed below and referring now to the drawings and figures:

FIG. 1 is an elevated perspective view, in reduced scale, of an embodiment of the resilient compressive surgical retractor securing device with capture assembly of the present invention attached to the surgical accessory bar of a halo style head fixation device;

FIG. 2 is a detailed elevated perspective view, in enlarged scale, of an embodiment of the resilient compressive surgical retractor securing device with capture assembly of the present invention;

FIG. 3 is a detailed elevated exploded perspective view, in enlarged scale, of an embodiment of the resilient compressive surgical retractor securing device with capture assembly of the present invention;

FIG. 4 is a detailed elevated exploded perspective view, in enlarged scale, of an embodiment of the resilient compressive surgical retractor securing device with capture assembly of the present invention;

FIG. 5 is an elevation view, in enlarged scale, of an embodiment of the resilient compressive surgical retractor securing device with capture assembly of the present invention showing the capture assembly with motion toward engagement of the retractor indicated;

FIG. 6 is an elevation view, in enlarged scale, of an embodiment of the resilient compressive surgical retractor securing device with capture assembly of the present invention showing the capture assembly with motion to disengagement of the retractor indicated;

FIG. 7 is an elevation view of an embodiment of the instant invention, not to scale;

FIG. 8 is an elevation view of an alternate embodiment of the instant invention, not to scale;

FIG. 9 is an elevation view of an alternate embodiment of the instant invention, not to scale; and

FIG. 10 is an elevation view of an alternate embodiment of the instant invention.

### DETAILED DESCRIPTION OF THE INVENTION

The surgical retractor securing device with capture assembly (100) of the instant invention enables a significant advance in the state of the art. The preferred embodiments of the device accomplish this by new and novel arrangements of elements and methods that are configured in unique and novel ways and which demonstrate previously unavailable but preferred and desirable capabilities. The detailed description set forth below in connection with the drawings is intended merely as a description of the presently preferred embodiments of the invention, and is not intended to represent the only form in which the present invention may be constructed or utilized. The description sets forth the designs, functions, means, and methods of implementing the invention in connection with the illustrated embodiments. It is to be understood, however, that the same or equivalent functions and features may be accomplished by different embodiments that are also intended to be encompassed within the spirit and scope of the invention.

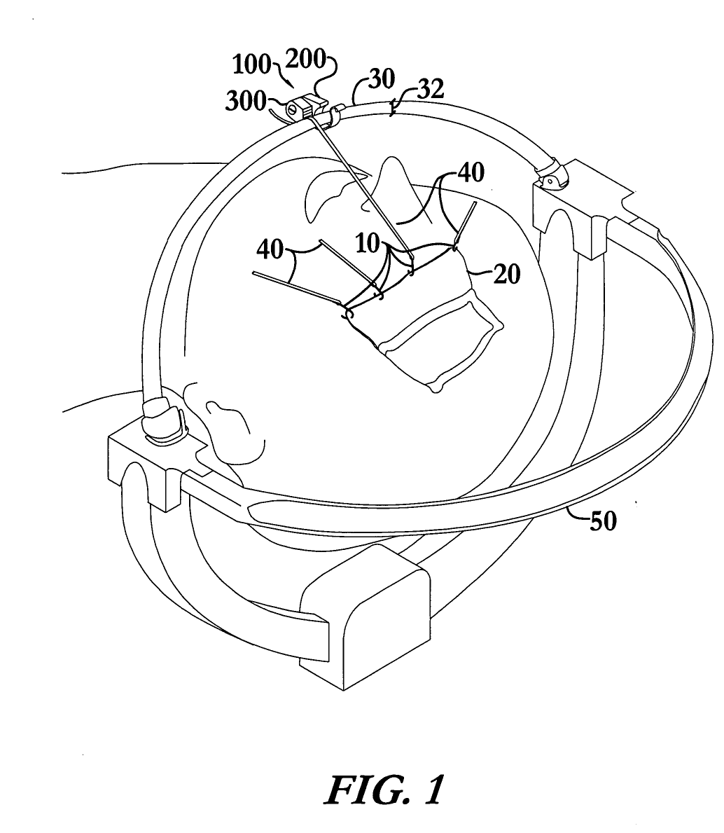

With reference generally now to FIGS. 1 through 10, the present invention is a surgical retractor securing device with capture assembly (100). The device may generally be described as having a compressive securing device (200) and a capture assembly (300).

In one embodiment, as seen in FIG. 1, a surgical retractor securing device with capture assembly (100) is adapted to be releasably secured to a surgical accessory bar (30). Such bars are well known in the art, and the bars have a surgical accessory bar width (32). The surgical retractor securing device with capture assembly (100) is operative to releasably retain an elasticized band (40) in cooperation with the surgical accessory bar (30). As shall be used and intended throughout the disclosure, the term “elasticized” is intended to represent materials that are fully elasticized, partially elasticized, or non-elasticized but that depend on external elasticity, such as, by way of example only, a spring, band or other external device, and also to include elasticity imparted by the retracted tissue itself.

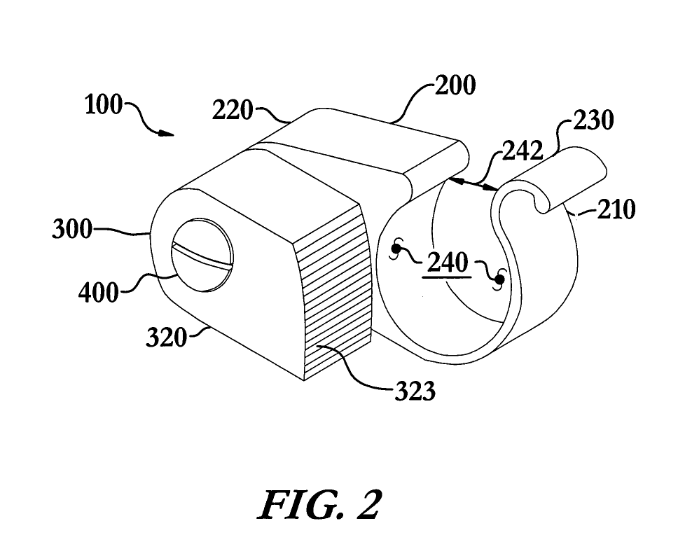

The surgical retractor securing device with capture assembly (100), as seen well in FIGS. 2-4, includes a securing device (200) for releasably joining the surgical retractor securing device with capture assembly (100) to the surgical accessory bar (30). The securing device (200) has a proximal end (210), a distal end (220), and an arm (230) adjacent the proximal end (210). The arm (230) may be configured to form a neck (240) between the arm (230) and the distal end (220). The width of the neck (240) is smaller in at least one dimension than the surgical accessory bar width (32) and the neck (240) has a neck opening (242) less than the surgical accessory bar width (32). Accordingly, the arm (230) must be displaced from a rest position to allow the surgical accessory bar (30) to pass through the neck opening (242) into the neck (240). The arm (230) therefore applies compressive force to the surgical accessory bar (30) to hold the surgical accessory bar (30) in the neck (240) when the surgical accessory bar (30) is in the securing position.

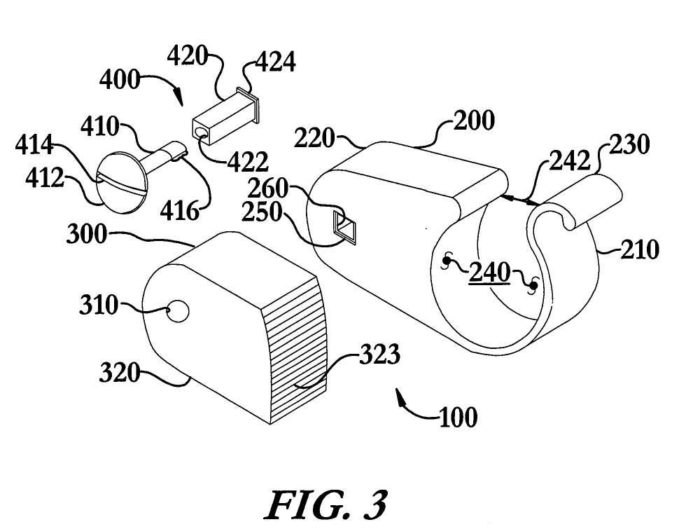

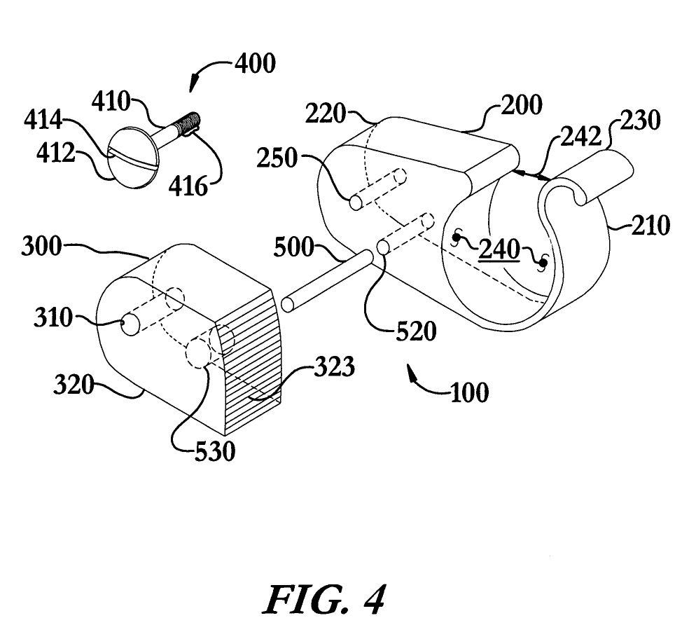

The surgical retractor securing device with capture assembly (100), seen assembled in FIG. 2 and disassembled in FIGS. 3-4, includes a capture assembly (300) for releasably retaining the elasticized band (40). The capture assembly (300) is rotatably attached to the securing device (200).

In one embodiment, seen well in FIGS. 2-8 and FIG. 10, the arm (230) is formed integrally to the securing device (200) and compressive force is created by the resilience of the arm (230) produced by the displacement of the arm (230) from the rest position.

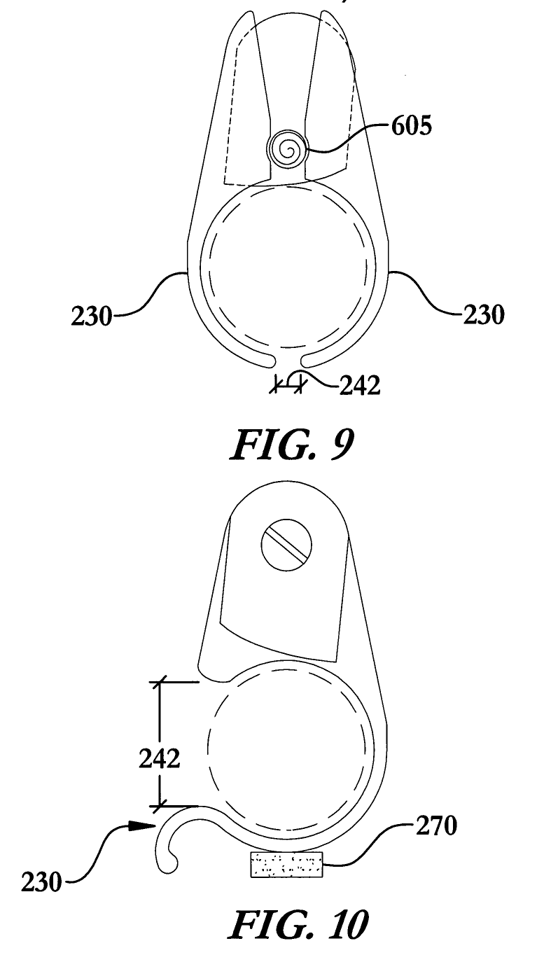

In another embodiment, seen well in FIG. 9, the arm (230) is releasably attached to the securing device (200) and compressive force is created by a force producing element (605) applying force to the arm (230). Such embodiment may be generally described as clip-like and the compressive force may be applied by springs, elastic bands, or a variety of other force producing elements as would be known by one skilled in the art.

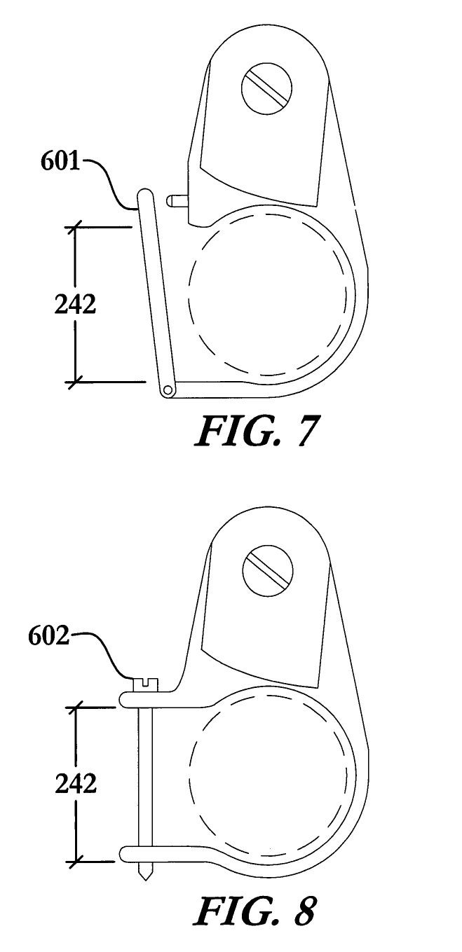

In addition to the force applied by the arm (230), additional means may be employed to retain the apparatus (100) on the surgical accessory bar (30). In one embodiment, seen in FIGS. 7-8, the apparatus (100) may have a releasable neck closure (600) attachable to the arm (230), and having an open position and a closed position, to close the neck opening (242) and secure the surgical accessory bar (33) in the neck (240). The releasable neck closure (600) is opened to mount or dismount the apparatus (100) on the surgical accessory bar (30), and when closed, the releasable neck closure (600) helps retain the apparatus in operative position. In various embodiments, the releasable neck closure (600) may be a releasable gate (601), or a releasable shackle (602), again seen in FIGS. 7-8, and one skilled in the art will be able to envision multiple other functional designs for such a releasable closure that may serve to further tighten and even lock the apparatus to the surgical accessory bar (30).

In a preferred embodiment, seen in FIGS. 3-6, the capture assembly (300) is formed with a capture pivot assembly receiver (310) and the securing device (200) is formed with a device pivot assembly receiver (250), and the capture assembly (300) is rotatably attached to the securing device (200) by a pivot assembly (400). Also as well seen in FIGS. 3-6, the capture assembly (300) may be formed with at least one gripping cam (320).

It may be desirable to limit the degree of rotation of the capture assembly (300) around the securing device (200), as therefore in one embodiment, the securing device (200) has a stop pin (500) and the capture assembly (300) has a stop pin limiter (530). Cooperation between the stop pin (500) and the stop pin limiter (530) limits an angular rotation of the capture assembly (300) about the securing device (200) to less than 45 degrees of rotational motion. In one particular embodiment, meant for illustration only, and seen in FIG. 4, the stop pin limiter (530) is a female receiver sized slightly somewhat larger than the male stop pin (500). Thus, varying the degree of oversize between these two structures allows the fabricator to vary the degree of angular rotation allowed. One skilled in the art will realize that the relative positions of these structures may be varied, and it is equally possible that the securing device (200) have a stop pin limiter (530) and for the capture assembly (300) to have a stop pin (500). In one particular embodiment, decreasing the degree of oversize may limit angular rotation of the capture assembly (300) about the securing device (200) to less than 15 degrees of rotational motion. Such limitations of rotation prevent the capture assembly (300) from rotating an excessive distance away form the elasticized band (40). In some embodiments, the stop pin (500) may be attached to the securing device (200) by means of the stop pin (500) being received into a stop pin receiver (520), as seen in FIG. 4, while in other embodiments, the stop pin (500) may be integrally formed with the securing device (200). Again, the relative position of the stop pin (500) and the stop pin receiver (520) may vary.

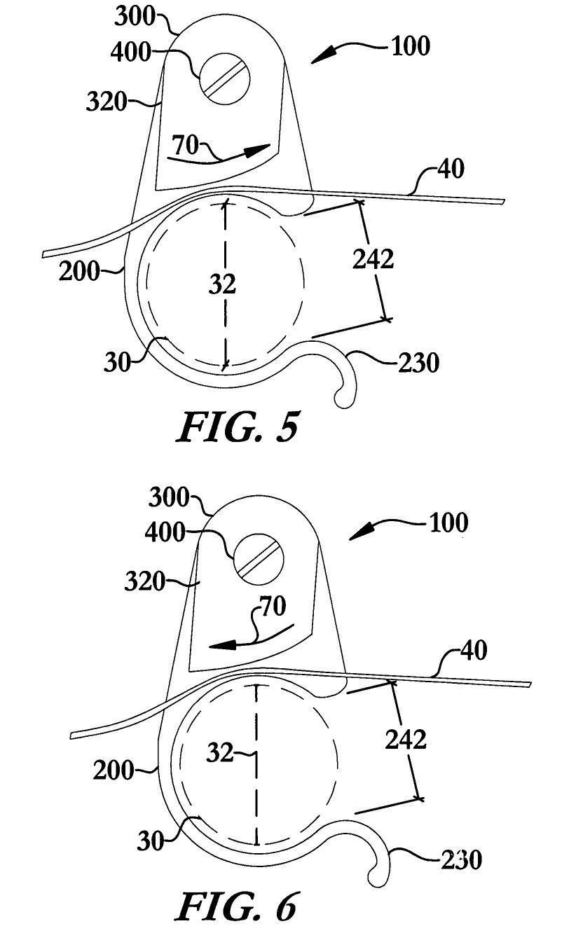

As may be well seen in FIGS. 2-4, and as well seen in conjunction with the elasticized band (40) in FIGS. 5-6, the capture assembly (300) may be a gripping cam (320) that releasably retains the elasticized band (40) between the surgical accessory bar (30) and the gripping cam (320). The gripping cam (320) may be rotatably attached to the securing device (200) by the pivot assembly (400) passing through a portion of the capture pivot assembly receiver (310) and a portion of the device pivot assembly receiver (250), seen well in FIGS. 3-4.

As seen well in FIGS. 2-4, in some embodiments, the gripping cam (320) may include a high friction gripping surface (323) to further aid in releasably retaining the elasticized band (40) by compressing the elasticized band between the gripping cam (320) and the surgical accessory bar (30). As would be well known to one skilled in the art, such a high friction gripping surface (323) may include a raised or incised pattern inscribed on the surface of interaction between the gripping cam (320) and the elasticized band (40).

Different functional methods may be employed in different embodiments to secure the capture assembly (300) to the securing device (200) in a rotable manner. In one embodiment, seen well in FIG. 3, the pivot assembly (400) may be formed of a fastener and stud type arrangement, in which a cam pivot pin (410) is formed with an enlarged head (412), a tool engager (414), and a threaded stud engager (416), and where the cam pivot pin (410) passes through a portion of the capture pivot assembly receiver (310). To secure the cam pivot pin (410), a stud (420) may be formed with a stud base plate (424) and a threaded receiver (422). The stud (420) may pass through a portion of the device pivot assembly receiver (250) in a direction retrograde to the cam pivot pin (410) and the threaded stud engager (416) may releasably mate to the threaded receiver (422) to hold the components together.

In another embodiment, seen well in FIG. 4, the cam pivot pin (410) is a shoulder bolt releasably attachable to the device pivot assembly receiver (250). The shoulder bolt ensures that the cam pivot pin (410) is tightened only enough to lightly hold the gripping cam (320) against the compressive securing device (200), and thus prevents over tightening.

Various means may be used to improve the releasable attachment of the surgical retractor securing device with capture assembly (100) to the surgical accessory bar. In one embodiment, by means of illustration, the securing device (200) has a magnet (270) in magnetic proximity to the neck (240), which tends to magnetically hold the securing device (200) to the surgical accessory bar (30).

Numerous alterations, modifications, and variations of the preferred embodiments disclosed herein will be apparent to those skilled in the art and they are all anticipated and contemplated to be within the spirit and scope of the instant invention. For example, although specific embodiments have been described in detail, those with skill in the art will understand that the preceding embodiments and variations can be modified to incorporate various types of substitute and or additional or alternative materials, relative arrangement of elements, and dimensional configurations. Accordingly, even though only few variations of the present invention are described herein, it is to be understood that the practice of such additional modifications and variations and the equivalents thereof, are within the spirit and scope of the invention as defined in the following claims.

We claim:

1. A surgical retractor securing device with capture assembly (100) adapted to be releasably secured to a surgical accessory bar (30) having a surgical accessory bar width (32) and operative to releasably retain an elasticized band (40), comprising:
(i) a securing device (200) for releasably joining the surgical retractor securing device with capture assembly (100) to the surgical accessory bar (30), the securing device (200) having a proximal end (210), a distal end (220), and an arm (230) adjacent the proximal end (210), wherein the arm (230) is configured to form a neck (240) between the arm (230) and the distal end (220) wherein the neck (240) is smaller in at least one dimension than the surgical accessory bar width (32), the neck (240) having a neck opening (242) wherein the neck opening (242) is less than the surgical accessory bar width (32) so that the arm (230) must be displaced from a rest position to allow the surgical accessory bar (30) to pass through the neck opening (242) into the neck (240), and the arm (230) applies compressive force to the surgical accessory bar (30) to hold the surgical accessory bar (30) in the neck (240) when the surgical accessory bar (30) is in the securing position; and
(ii) a capture assembly (300) for releasably retaining the elasticized band (40), wherein the capture assembly (300) is rotatably attached to the securing device (200).

2. The apparatus according to claim 1, wherein the arm (230) is formed integrally to the securing device (200) and compressive force is created by the resilience of the arm (230) produced by the displacement of the arm (230) from the rest position.

3. The apparatus according to claim 1, wherein the arm (230) is releasably attached to the securing device (200) and compressive force is created by a force producing element (605) applying force to the arm (230).

4. The apparatus according to claim 1, further comprising a releasable neck closure (600) attachable to the arm (230) having an open position and a closed position, to close the neck opening (242) and secure the surgical accessory bar (32) in the neck (240).

5. The apparatus according to claim 4, wherein the releasable neck closure (600) is selected from the group of closures consisting of a releasable gate (601) and a releasable shackle (602).

6. The apparatus according to claim 1, wherein the capture assembly (300) is formed with a capture pivot assembly receiver (310) and the securing device (200) is formed with a device pivot assembly receiver (250), and the capture assembly (300) is rotatably attached to the securing device (200) by a pivot assembly (400).

7. The apparatus according to claim 1, wherein the capture assembly (300) is formed with at least one gripping cam (320).

8. The apparatus according to claim 1, wherein the securing device (200) has a stop pin (500) and the capture assembly (300) has a stop pin limiter (530) and cooperation between the stop pin (500) and the stop pin limiter (530) limits an angular rotation of the capture assembly (300) about the securing device (200) to less than 45 degrees of rotational motion.

9. The apparatus according to claim 1, wherein the securing device (200) has a stop pin limiter (530) and the capture assembly (300) has a stop pin (500) and cooperation between the stop pin (500) and the stop pin limiter (530) limits an angular rotation of the capture assembly (300) about the securing device (200) to less than 45 degrees of rotational motion.

10. The apparatus according to claim 1, wherein the securing device (200) has a stop pin (500) and the capture assembly (300) has a stop pin limiter (530) and cooperation between the stop pin (500) and the stop pin limiter (530) limits an angular rotation of the capture assembly (300) about the securing device (200) to less than 15 degrees of rotational motion.

11. The apparatus according to claim 1, wherein the securing device (200) has a stop pin limiter (530) and the capture assembly (300) has a stop pin (500) and cooperation between the stop pin (500) and the stop pin limiter (530) limits an angular rotation of the capture assembly (300) about the securing device (200) to less than 15 degrees of rotational motion.

12. The apparatus according to claim 6, wherein the capture assembly (300) is a gripping cam (320) that releasably retains the elasticized band (40) between the surgical accessory bar (30) and the gripping cam (320), and the gripping cam (320) is rotatably attached to the securing device (200) by the pivot assembly (400) passing through a portion of the capture pivot assembly receiver (310) and a portion of the device pivot assembly receiver (250).

13. The apparatus according to claim 7, wherein the gripping cam (320) includes a high friction gripping surface (323) releasably retaining the elasticized band (40) by compressing the elasticized band between the gripping cam (320) and the surgical accessory bar (30).

14. The apparatus according to claim 12, wherein the pivot assembly (400) comprises:
(i) a cam pivot pin (410), wherein the cam pivot pin (410) is formed with an enlarged head (412), a tool engager (414), and a threaded stud engager (416) and wherein the cam pivot pin (410) passes through a portion of the capture pivot assembly receiver (310);
(ii) a stud (420), wherein the stud (420) is formed with a stud base plate (424) and a threaded receiver (422) and passes the stud (420) passes through a portion of the device pivot assembly receiver (250) in a direction retrograde to the cam pivot pin (410); and
(iii) the threaded stud engager (416) releasably mates to the threaded receiver (422).

15. The apparatus according to claim 14, wherein the cam pivot pin (410) is a shoulder bolt releasably attachable to the device pivot assembly receiver (250).

16. The apparatus according to claim 1, wherein the securing device (200) further comprises a magnet (270) in magnetic proximity to the neck (240).

17. A surgical retractor securing device with capture assembly (100) adapted to be releasably secured to a surgical accessory bar (30) having a surgical accessory bar width (32) and operative to releasably retain an elasticized band (40), comprising:
(i) a securing device (200) for releasably joining the surgical retractor securing device with capture assembly (100) to the surgical accessory bar (30), the securing device (200) having a proximal end (210), a distal end (220), and an arm (230) adjacent the proximal end (210), wherein the arm (230) is configured to form a neck (240) between the arm (230) and the distal end (220) wherein the neck (240) is smaller in at least one dimension than the surgical accessory bar width (32), the neck (240) having a neck opening (242) wherein the neck opening (242) is less than the surgical accessory bar width (32) so that the arm (230) must be displaced from a rest position to allow the surgical accessory bar (30) to pass through the neck opening (242) into the neck (240), and the arm (230) applies compressive force to hold the surgical accessory bar (30) in the neck (240) when the surgical accessory bar (30) is in the securing position, the arm (230) being formed integrally to the securing device (200) and compressive force is created by the resilience of the arm (230) produced by the displacement of the arm (230) from the rest position; and
(ii) a capture assembly (300) for releasably retaining the elasticized band (40), wherein the capture assembly (300) is rotatably attached to the securing device (200).

18. The apparatus according to claim 17, further comprising a releasable neck closure (600) attachable to the arm (230) having an open position and a closed position, to close the neck opening (242) and secure the surgical accessory bar (32) in the neck (240).

19. The apparatus according to claim 17, wherein the securing device (200) has a stop pin (500) and the capture assembly (300) has a stop pin limiter (530) and cooperation between the stop pin (500) and the stop pin limiter (530) limits an angular rotation of the capture assembly (300) about the securing device (200) to less than 45 degrees of rotational motion.

20. The apparatus according to claim 17, wherein the capture assembly (300) is a gripping cam (320) that releasably retains the elasticized band (40) between the surgical accessory bar (30) and the gripping cam (320), and the gripping cam (320) is rotatably attached to the securing device (200) by a pivot assembly (400) passing through a portion of the capture pivot assembly receiver (310) and a portion of the device pivot assembly receiver (250).

21. The apparatus according to claim 20, wherein pivot assembly (400) is a shoulder bolt releasably attachable to the device pivot assembly receiver (250).

22. A surgical retractor securing device with capture assembly (100) adapted to be releasably secured to a surgical accessory bar (30) having a surgical accessory bar width (32) and operative to releasably retain an elasticized band (40), comprising:
(i) a securing device (200) for releasably joining the surgical retractor securing device with capture assembly (100) to the surgical accessory bar (30), the securing device (200) having a proximal end (210), a distal end (220), and an arm (230) adjacent the proximal end (210), wherein the arm (230) is configured to form a neck (240) between the arm (230) and the distal end (220) wherein the neck (240) is smaller in at least one dimension than the surgical accessory bar width (32), the neck (240) having a neck opening (242) wherein the neck opening (242) is less than the surgical accessory bar width (32) so that the arm (230) must be displaced from a rest position to allow the surgical accessory bar (30) to pass through the neck opening (242) into the neck (240), the arm (230) applies compressive force to hold the surgical accessory bar (30) in the neck (240) when the surgical accessory bar (30) is in the securing position and wherein the arm (230) is releasably attached to the securing device (200) and compressive force is created by a force producing element (605) applying force to the arm (230); and
(ii) a capture assembly (300) for releasably retaining the elasticized band (40), wherein the capture assembly (300) is rotatably attached to the securing device (200).

23. The apparatus according to claim 22, further comprising a releasable neck closure (600) attachable to the arm (230) having an open position and a closed position, to close the neck opening (242) and secure the surgical accessory bar (32) in the neck (240).
